(19) United States
(12) Patent Application Publication
(10) Pub. No.: US 2012/0045413 A1
(43) Pub. Date: Feb. 23, 2012

(54) Human papilloma virus peptide-specific T-cell response for treatment of warts

(76) Inventors: Mayumi Nakagawa, Little Rock, AR (US); Kevin Kim, Saint Louis, MO (US); Thomas Horn, Brookline, MA (US)

(21) Appl. No.: 13/136,801

(22) Filed: Aug. 11, 2011

Related U.S. Application Data
(63) Continuation-in-part of application No. 13/066,128, filed on Apr. 7, 2011, now abandoned.
(60) Provisional application No. 61/342,174, filed on Apr. 9, 2010.

(51) Int. Cl. A61K 39/12; C07K 7/08; C07K 7/06; A61P 31/20; A61K 38/19; A61K 38/21; A61K 38/20; A61P 35/00; C07K 14/025; A61K 39/165

(52) U.S. Cl. 424/85.2; 530/324; 530/325; 530/326; 530/327; 530/328; 424/186.1; 424/85.1; 424/85.7; 424/85.6; 424/85.5

(57) ABSTRACT

The inventors have treated human patients for warts by intralesional injection of Candida antigen to induce a delayed-type hypersensitivity response in the patients. This creates an immune response that recognizes the antigens of the human papilloma virus (HPV) found in and causing the warts. It was found that the patients showed a response to HPV type 57 L1 peptide 380-412 (a peptide consisting of amino acid residues 380-412 of the protein L1) and HPV type 57 protein E4 (E4 10-30). One embodiment of the invention provides a pharmaceutical composition comprising a polypeptide comprising (a) L1 380-412 (SEQ ID NO:3) or a fragment of at least 8 residues of SEQ ID NO:3 or (b) E4 10-30 (SEQ ID NO:4) or a fragment of at least 8 residues of E4 10-30 (SEQ ID NO:4), wherein the composition is immunogenic in humans.

This application is a continuation-in-part of U.S. utility patent application Ser. No. 13/066,128, filed Apr. 7, 2011, which claims priority under 35 U.S.C. 119(e) from provisional patent application No. 61/342,174, filed Apr. 9, 2010.

### BACKGROUND

Warts are benign epidermal tumors caused by human papillomaviruses (HPV). There are more than 100 distinct HPV types that have been isolated from cutaneous and mucosal lesions. Depending on the site of infection and the HPV type, cutaneous manifestation of HPV infections present clinically in different forms. Common warts or verrucae vulgaris represent about 70% of cutaneous lesions (1). The closely related HPV types 2, 27, and 57 predominantly cause common warts which appear as dome-shaped papules or nodules most often on the knees, elbows, fingers, and hands (2,3). Common warts affect people of all ages, but are most prevalent in children and young adults; it is estimated to occur in up to 10% of children and young adults (4). Fortunately, most warts are self-limited and regress spontaneously within 2 years (5). Most patients seek medical treatment prior to spontaneous resolution, however, due to discomfort and embarrassment. It is estimated that warts account for 8% of visits to dermatologists (6).

The treatment for warts is a challenge because no one treatment is uniformly effective. It is common for an individual to undergo many different types of therapies before finding one that is effective. The current recommended first-line therapies for common warts are topical salicylic acid and cryotherapy with liquid nitrogen. The recommended second-line therapy is Cantharidin and third-line therapies include Bleomycin (Blenoxane) and pulsed dye laser therapy (4). These treatments have variable results, are oftentimes painful, and require treatment of individual warts. In addition, recurrence rates are unfortunately high (39%) for cryotherapy (7). The high recurrence rates may be due to the sub-optimal level of immunity to HPV. Therefore, the goal of new therapies is to utilize and enhance the immune response against HPV, which may reduce the number of warts that are treated since a systemic immune response may be able to resolve all warts, treated and untreated alike. In addition, enhanced HPV specific T-cell response may give the patient long-standing immunity and prevent recurrence.

It is well established that cell-mediated immune response plays a major role in controlling HPV infections (8). This is evidenced by the increased susceptibility to HPV infection in immunosuppressed individuals (9-11) and the development of warts in 90% of renal-transplant recipients within five years after transplantation (12). Therefore, therapy modalities such as immunotherapy have been employed to activate the immunological response to HPV. One method of immunotherapy is the intralesional injection of skin test antigens such as Candida, mumps, and Trichophyton. Several studies have shown the treatment of warts with Candida, mumps, and/or Trichophyton antigen injection to be effective in not only resolving treated warts but also distant untreated warts (13-16). Other studies have also shown the effectiveness of Candida antigen injection immunotherapy in the pediatric populations (14-18).

The use of recall antigens for treating warts is not yet Food and Drug Administration (FDA) approved. The primary goal of this work was to assess the safety of Candin® as an investigational new drug (IND) for the treatment of warts. In addition, clinical resolution of treated and untreated warts was evaluated and immunological responses were examined using an ex vivo interferon-γ enzyme-linked immunospot (IFN-γ ELISPOT) assay in order to elucidate the immunological mechanisms behind the successful regression of warts in patients undergoing Candin® injection immunotherapy.

### SUMMARY

The inventors have treated human patients for warts by intralesional injection of Candida antigen (CANDIN®). The Candida antigen induces a delayed-type hypersensitivity response in the patients. This appears to create an immune response that recognizes the antigens of the human papilloma virus found in and causing the warts, because not only the treated wart but other distant warts in the same patient resolve after treatment in most patients. In addition, the inventors have found that six of 10 patients examined showed an immune response to HPV type 57 L1 peptide 380-412 (a peptide consisting of amino acid residues 380-412 of the protein L1). Another patient developed an immune response to a peptide of the HPV type 57 protein E4 (E4 10-30). Thus, L1 and E4, and particularly L1 380-412 and E4 10-30, appear to be particularly suited as targets for the anti-HPV immune response, and inclusion of these proteins and peptides of the proteins in immunogenic formulations to treat warts and other epithelial tumors caused by HPV can increase the effectiveness of the treatment.

One embodiment of the invention provides a pharmaceutical composition comprising a polypeptide comprising (a) L1 380-412 (SEQ ID NO:3) or a fragment of at least 8 residues of SEQ ID NO:3 or (b) E4 10-30 (SEQ ID NO:4) or a fragment of at least 8 residues of E4 10-30 (SEQ ID NO:4), wherein the polypeptide does not comprise L1 (SEQ ID NO:1) or E4 (SEQ ID NO:2), wherein the composition is immunogenic in humans.

Another embodiment provides a pharmaceutical composition comprising (a) an antigen not found in human papilloma virus and (b) (i) a polypeptide comprising human papilloma virus L1 (SEQ ID NO:1) or an antigenic fragment of SEQ ID NO:1 or (b) (ii) a polypeptide comprising HPV E4 (SEQ ID NO:2) or an antigenic fragment of SEQ ID NO:2; wherein the composition is capable of treating a benign epithelial tumor caused by a human papilloma virus. Preferably the composition is capable of inducing a delayed-type hypersensitivity (DTH) response in humans.

Another embodiment provides a method of treating an epithelial tumor in a human comprising: inoculating the human with a pharmaceutical composition comprising a polypeptide comprising (a) L1 380-412 (SEQ ID NO:3) or a fragment of at least 8 residues of SEQ ID NO:3 or (b) E4 10-30 (SEQ ID NO:4) or a fragment of at least 8 residues of SEQ ID NO:4, wherein the polypeptide does not comprise L1 (SEQ ID NO:1) or E4 (SEQ ID NO:2), wherein the composition is immunogenic in humans; wherein the epithelial tumor is caused by a human papilloma virus.

Another embodiment of the invention provides a kit comprising (a) a pharmaceutical composition, suitable for inoculation of a human, comprising an antigen capable of inducing a delayed type hypersensitivity response in humans, wherein the composition is immunogenic, wherein the antigen is not found in a human papilloma virus; and (b) a pharmaceutical composition, suitable for inoculation into a human, comprising (i) a polypeptide comprising human papillomavirus (HPV) L1 (SEQ ID NO:1) or an antigenic fragment of SEQ ID NO:1 or (ii) a polypeptide comprising HPV E4 (SEQ ID NO:2) or an antigenic fragment of SEQ ID NO:2.

### DETAILED DESCRIPTION

The inventors conducted a Phase I clinical trial of treatment of warts by intralesional injection of Candida antigen (Example 1 below). Eighteen patients with at least two cutaneous non-genital non-facial warts were recruited. Eleven completed the protocol. Patients received an intralesional injection of Candida antigen into the largest wart every three weeks until complete resolution of the treated wart occurred or for a maximum of 10 treatments or for 5 treatments if less than 25% regression observed (non-responder). Eleven of the 18 patients completed the study, 9 (82%) of whom had complete resolution of their treated wart.

To test immune responses against HPV antigens, several peptides from HPV proteins were selected that were predicted to bind promiscuously to multiple HLA alleles within HLA A2 and HLA DR supertypes. Peptides that bind to multiple HLA alleles can be used to immunize a large fraction of the population. Peptides were selected from proteins of the most common HPV types detected in warts of patients previously tested (HPV 2a, 27, and 57). HPV 57 E1 (231-260 and 251-286), E2 (188-208), E4 (10-30), E6 (17-55), and L1 (380-412), were chosen.

Peripheral blood mononuclear cells (PBMC) of patients in the study were collected before the first vaccination and after completing treatment. The pre- and post-vaccination PBMC samples were tested by ELISPOT assay for reaction against each of the peptides.

Six of 10 patients examined (all of the six were complete or partial responders) had a positive response to HPV type 57 L1 (380-412) peptide. Five of the six developed the response to L1 (380-412) after treatment with Candida antigen. One of these six also developed a response to HPV type 57 E4 (10-30) peptide.

Thus, L1 and E4 are suitable proteins to include in a vaccine to treat warts, and especially peptides L1 (380-412) and E4 (10-30). They may be included in a composition containing an antigen unrelated to HPV that induces a delayed type hypersensitivity response, such as Candida antigen, mumps antigen, or trichophyton antigen. Or the peptides or proteins in other embodiments can be the only antigenic components of the vaccine or included in a vaccine with other HPV antigens.

A first embodiment of the invention provides a pharmaceutical composition comprising a polypeptide comprising (a) L1 380-412 (SEQ ID NO:3) or a fragment of at least 8 residues of SEQ ID NO:3 or (b) E4 10-30 (SEQ ID NO:4) or a fragment of at least 8 residues of E4 10-30 (SEQ ID NO:4), wherein the polypeptide does not comprise L1 (SEQ ID NO:1) or E4 (SEQ ID NO:2), wherein the composition is immunogenic in humans.

Another embodiment provides a pharmaceutical composition comprising (a) an antigen not found in human papilloma virus and (b) (i) a polypeptide comprising human papilloma virus L1 (SEQ ID NO:1) or an antigenic fragment of SEQ ID NO:1 or (b) (ii) a polypeptide comprising HPV E4 (SEQ ID NO:2) or an antigenic fragment of SEQ ID NO:2; wherein the composition is capable of treating a benign epithelial tumor caused by a human papilloma virus.

The polypeptides may be optionally amidated at the C-terminus or acetylated at the N-terminus, or both. Each of these modifications may stabilize the peptide.

In a particular embodiment, the polypeptides are amidated at the C-terminus. In a particular embodiment, the polypeptide is or comprises L1 380-412 (SEQ ID NO:3) and is amidated at the C terminus.

Some or all of the polypeptides in the pharmaceutical composition may be present as dimers linked through cysteine disulfides. This can be a more stable formulation for storage. It is particularly useful where the polypeptide contains exactly one cysteine residue, so there is only one possible configuration of disulfide.

Thus, in particular embodiments of the pharmaceutical composition, the polypeptide is dimerized by cysteine disulfides. In particular embodiments, the polypeptide has exactly one cysteine residue and is dimerized through the cysteine residue in a disulfide. In particular embodiments, the comprises L1 380-412 (SEQ ID NO:3) dimerized through the cysteine residue of L1 380-412.

The polypeptides of the invention in the pharmaceutical compositions for treating an HPV epithelial tumor are preferably fragments of HPV. Small peptides can be chemically synthesized, which avoids contamination with, for instance, E. coli components if the protein were produced recombinantly in E. coli.

In particular embodiments, the polypeptide comprising an antigenic fragment of L1 or E4 is a polypeptide of 8-100 amino acid residues, 8-80, 8-60, 8-50, amino acid residues, 8, 9, or 10 amino acid residues. In specific embodiment, the polypeptide is a polypeptide of no more than 100, no more than 80, no more than 60, no more than 50, no more than 40, or no more than 30 amino acid residues. In specific embodiments, it is a polypeptide of at least 8, at least 9, at least 10, at least 12, at least 15, at least 20, or at least 25 amino acid residues.

In specific embodiments, the polypeptide comprises SEQ ID NO:3 or a fragment of at least 8 residues of SEQ ID NO:3.

In specific embodiments, the polypeptide comprises SEQ ID NO:4 or a fragment of at least 8 residues of SEQ ID NO:4.

In specific embodiments, the compositions comprise both (i) a polypeptide comprising L1 or an antigenic fragment of L1 and (ii) a polypeptide comprising E4 or an antigenic fragment of E4. For instance, in specific embodiments, the composition may comprise (i) SEQ ID NO:3 or an antigenic fragment of at least 8 residues of SEQ ID NO:3 and (ii) SEQ ID NO:4 or an antigenic fragment of at least 8 residues of SEQ ID NO:4. The E4 and L1 antigens could also be on the same polypeptide.

The composition preferably is capable of treating a benign epithelial tumor caused by a human papilloma virus.

In specific embodiments, the composition comprises one or more antigens not found in a human papilloma virus. The non-HPV antigen is preferably a protein antigen. The one or more non-HPV antigens are preferably capable of inducing a cutaneous delayed-type hypersensitivity response in humans when injected intradermally.

Skin test antigens are suitable for the composition. These are capable of inducing a cutaneous DTH response.

Preferably the non-HPV antigens are antigens that a large fraction of the population already has immune recognition to. These will induce a DTH response upon the first treatment injection. Antigens to which a patient is not already sensitized, if the antigens are capable of inducing a DTH response, can induce a DTH response in second and subsequent injections more than 1-2 weeks after the first sensitizing injection.

In specific embodiments, the non-HPV antigen is a candida antigen, a trichophyton antigen, or a mumps antigen. In other embodiments, the composition comprises a combination of two or three of these antigens.

In particular embodiments, the non-HPV antigen is a bacterial, a fungal, or a viral antigen. In some embodiments, the composition comprises a combination of two or more of a bacterial, a fungal, or a viral antigen.

In particular embodiments, the compositions comprise non-HPV antigens from two or more different species (e.g., bacterial, fungal, or viral species).

The antigens in specific embodiments are protein antigens.

The antigenic fragments of L1 or E4 also preferably are capable of inducing a delayed-type hypersensitivity response in humans. Preferably they are capable of inducing a cutaneous DTH response in at least 50%, at least 70%, or at least 90%, of humans or nearly all humans who are not immunocompromised.

The pharmaceutical compositions should be sterile and suitable for injection intradermally, intramuscularly, or subcutaneously, or sterile and suitable for nasal or vaginal inoculation.

Certain cytokines and colony stimulating factors are known to enhance a DTH response and anti-viral immune responses. Human interferon-α is known to be useful in the treatment of several viral infections, including chronic hepatitis B virus and herpes zoster. U.S. Pat. No. 5,165,921 discloses treating condyloma acuminatum, commonly referred to as genital warts, which are associated with papilloma viruses, with a topical formulation of interferon-α. Additionally, warts can also be treated by direct injection of interferon into the warts (16,17).

In some embodiment, the pharmaceutical compositions comprise at least one cytokine or colony stimulating factor. In specific embodiments, the composition comprises granulocyte macrophage colony stimulating colony stimulating factor, interferon alpha, interferon beta, interferon gamma, interleukin-2, or interleukin-12.

The pharmaceutical compositions described herein and antigens of the pharmaceutical compositions described herein are preferably capable of inducing a DTH response in humans. A composition or antigen that is “capable of inducing a DTH response in humans” is one which when injected twice intradermally into humans, with a spacing of 2 to 3 weeks between the first and second injections, will cause a DTH response upon the second injection (where the first injection may sensitize the person to the antigen) in at least 50% of the population. To determine whether an antigen is capable of inducing a DTH response, the antigen should be injected intradermally at an appropriate concentration and with an adjuvant.

Another embodiment of the invention is a method of treating an epithelial tumor in a human comprising: inoculating the human with a pharmaceutical composition comprising a polypeptide comprising (a) L1 380-412 (SEQ ID NO:3) or a fragment of at least 8 residues of SEQ ID NO:3 or (b) E4 10-30 (SEQ ID NO:4) or a fragment of at least 8 residues of SEQ ID NO:4, wherein the polypeptide does not comprise L1 (SEQ ID NO:1) or E4 (SEQ ID NO:2), wherein the composition is immunogenic in humans; wherein the epithelial tumor is caused by a human papilloma virus.

The inoculating step may be by intradermal, intramuscular, or subcutaneous injection. These injections may also be intralesional. In other embodiments, the inoculating step is intravaginal or intranasal inoculation.

Preferably the step of inoculating the composition causes a DTH response in the human.

The method may involve inoculating the human with the pharmaceutical composition into the human two or more times, with the two or more inoculations spaced at least one week apart, more preferably at least two weeks apart.

The method preferably involves inoculating the human with a sufficient amount of the composition in a sufficient number of inoculations to be effective to cause regression of the epithelial tumor.

The method preferably also involves inoculating the human with at least one antigen not found in a human papilloma virus, wherein the antigen induces a DTH response in the human. The non-HPV antigen or antigens are preferably in the same pharmaceutical composition as the polypeptide comprising the L1 or E4 sequence. But the non-HPV antigen or antigens may be in a separate composition inoculated separately. Preferably they are injected in the same location so that the immune cells of the DTH response will be in the location of the polypeptide comprising the L1 or E4 sequences to enhance the immune response to the HPV protein sequences.

The method may involve injecting the composition or compositions intramuscularly, intradermally, or subcutaneously, or vaginal or nasal inoculation. Preferably, the injections are intralesional (intradermal).

In particular embodiments of the methods of treating an epithelial tumor, the epithelial tumor is a benign epithelial tumor.

In specific embodiments, the benign epithelial tumor is a verruca, a condyloma, a bowenoid populosis, a laryngeal papilloma, or a epidermodysplasia verruciformis.

In specific embodiments, the epithelial tumor is malignant. In specific embodiments, it is a tumor of a cervical carcinoma tumor, vaginal carcinoma tumor, or oral carcinoma tumor.

As stated above, the step of inoculating a human with the composition preferably induces a DTH response.

In specific embodiments, the method further comprises injecting at least one cytokine or colony stimulating factor into the human. The cytokine or colony stimulating factor may be in the same composition as the HPV protein sequences, and/or the same composition as the non-HPV antigens if those are in a separate composition. The cytokine or colony stimulating factor can also be in a separate composition from any of the antigens and can be administered separately from the antigens.

Further details of using injection of non-HPV antigens that cause a DTH response to treat an epithelial tumor caused by HPV can be found in U.S. Pat. No. 6,350,451.

One embodiment provides a method of treating an epithelial tumor in a human comprising: inoculating the human with a pharmaceutical composition comprising a polypeptide comprising (a) L1 380-412 (SEQ ID NO:3) or a fragment of at least 8 residues of SEQ ID NO:3 or (b) E4 10-30 (SEQ ID NO:4) or a fragment of at least 8 residues of SEQ ID NO:4, wherein the polypeptide does not comprise L1 (SEQ ID NO:1) or E4 (SEQ ID NO:2), wherein the composition is immunogenic in humans; wherein the epithelial tumor is caused by a human papilloma virus. The antigens (a) and (b) can both be present in the composition and both be on the same polypeptide or on separate polypeptides.

The invention will now be illustrated by the following example, which is intended to illustrate the invention without limiting its scope.

### EXAMPLES

### Example 1

### Phase I Clinical Trial of Intralesional Injection of Candida Antigen for the Treatment of Warts

It is well established that cell-mediated immune response plays a major role in controlling HPV infections (8). This is evidenced by the increased susceptibility to HPV infection in immunosuppressed individuals (9-11) and the development of warts in 90% of renal-transplant recipients within five years after transplantation (12). Therefore, therapy modalities such as immunotherapy have been employed to activate the immunological response to HPV. One method of immunotherapy is the intralesional injection of skin test antigens such as Candida, mumps, and Trichophyton. Several studies have shown the treatment of warts with Candida, mumps, and/or Trichophyton antigen injection to be effective in not only resolving treated warts but also distant untreated warts (13-16). Other studies have also shown the effectiveness of Candida antigen injection immunotherapy in the pediatric populations (14-18).

The primary goal of this work was to assess the safety of Candin® as an investigational new drug (IND) for the treatment of warts. In addition, clinical resolution of treated and untreated warts was evaluated and immunological responses were examined using an ex vivo interferon-γ enzyme-linked immunospot (IFN-γ ELISPOT) assay in order to elucidate the immunological mechanisms behind the successful regression of warts in patients undergoing Candin® injection immunotherapy.

### Methods

### Study Design and Patients

The study protocol was approved by the institutional review board of the University of Arkansas for Medical Sciences (UAMS), and the ClinicalTrials.gov identifier is NCT00569231. Patients for this study were recruited during the period of February 2007 and May 2009 from the outpatient Dermatology Clinic at UAMS. Informed consent was obtained from all participants. Patients' characteristics are summarized in Table 1. The inclusion criteria were being between 18 and 50 years old, having at least two cutaneous, non-genital, non-facial warts, and having a negative urine pregnancy test with an agreement to use two methods of birth-control for women of child-bearing potential. The exclusion criteria were iatrogenic immunosuppression, primary immunosuppression, pregnancy, lactation, allergy to thimerasol or the Candida antigens, history of asthma or diabetes, current use of non-selective beta blockers or H2 antagonists, history of keloid formation, history of alcohol or illicit drug abuse, and previous treatment with Candida antigens for warts. Patients who received treatments prior to the beginning of the study were enrolled after at a 30-day washout period.

TABLE 1
Patient Characteristics

| Patient No. | Age, y | Gender | Race | Prior Treatment(s) on Largest Wart | Location(s) of Wart |
|---|---|---|---|---|---|
| 1 | 28 | M | W | Cryotherapy; OTC | Palm of hand; finger |
| 2 | 36 | M | W | Cryotherapy; laser treatment; surgical removal | Finger |
| 3 | 27 | M | W | OTC | Finger |
| 4 | 30 | F | W | Cryotherapy | Dorsum of hand; finger |
| 5 | 19 | F | W | None | Bottom of foot; toe |
| 6 | 38 | M | W | Cryotherapy; scraping | Bottom of foot |
| 7 | 27 | F | W | Cryotherapy; laser treatment; surgical removal; acid; tar; scraping; Aldara ® | Bottom of foot; toe |
| 8 | 18 | F | W | Cryotherapy; Aldara ® | Knee; finger |
| 9 | 21 | F | B | Cryotherapy | Scalp |
| 10 | 46 | M | W | Cryotherapy; OTC; burning; immunological; acid; scraping | Finger |
| 11 | 25 | M | W | Cryotherapy; OTC; filing | Finger |
| 12 | 28 | F | W | OTC | Bottom of foot; toe |
| 13 | 25 | F | W | Cryotherapy; surgical removal; Aldara ® | Bottom of foot |
| 14 | 44 | M | W | Cryotherapy; OTC; Burning | Finger |
| 15 | 24 | M | W | OTC | Finger |
| 16 | 18 | M | W | Cryotherapy; surgical removal | Finger; elbow |
| 17 | 41 | M | B | Cryotherapy | Finger; toe |
| 18 | 41 | M | W | Cryotherapy; OTC; surgical removal; Aldara ® | Bottom of foot |

Abbreviations:
M, male;
F, female;
W, white;
B, black;
OTC, over the counter.

### Treatment

Each patient received an intralesional injection of 0.3 ml of Candida antigen, Candin® (Allermed Laboratories, San Diego, Calif.), into their largest wart at the baseline visit and then at each visit every three weeks +/−3 days. One wart was selected and treated in the same manner throughout the study. Injections were continued until complete resolution of the treated wart was achieved or until a maximum of 10 treatments were given or regression of less than 25% was observed after 5 injections (non-responder).

### Clinical Evaluation

The adverse events were graded according to the Common Terminology Criteria for Adverse events (CTCAE) version 3.0 toxicity grading scale: Grade 1 (mild), Grade 2 (moderate), Grade 3 (severe), and Grade 4 (potentially life threatening). The injected wart and up to three anatomically distant warts were measured at each visit. Surface area of warts was estimated by measuring the diameter. When the patient completed the protocol, response was determined by the overall percentage of the resolution from the initial visit. Patients were classified as ‘complete responders’ if they had complete resolution of the injected wart, ‘strong partial responders’ if the injected wart regressed between 75% and 99% in diameter, ‘partial responders’ between 25% and 75% in diameter, and ‘non-responders’ if they had not achieved at least 25% regression in diameter of the injected wart.

### HPV Typing

One shaved biopsy of a wart was obtained at the first visit from each patient for HPV-DNA testing. The sample wart tissue was snap frozen within 2.5 hours of biopsy and stored in liquid nitrogen storage tank. The frozen wart tissues were thawed by transferring to a microcentrifuge tube containing 250 μl of sterile deionized distilled water. For DNA extraction and purification, the QIAamp MinElute Media Kit (Qiagen Inc., Valencia, Calif.) was used according to manufacturer's instructions. Viral typing was then performed on the DNA extracted from the wart via polymerase chain reaction (PCR) to detect 1) HPV 2, 27, and/or 57 in a multiplex reaction, (19), 2) HPV 2a, 27, or 57 using a common primer (20), and 3) β-globin gene as a positive control (21). The PCR products were run on a 1.0% agarose gel with ethidium bromide in Tris-Borate-EDTA buffer and identified as a particular HPV genotype based on the appropriate size of the PCR product (19-21).

### ELISPOT Assay

At the baseline visit, 40 mL of venous blood was drawn from each patient prior to initiation of Candida injections. Once the patient achieved complete resolution of the treated wart or after the fifth injection of Candida, another 40 ml of venous blood was drawn from the patient. If the patient was still responding to treatment (>25% resolution), but had not achieved complete resolution after 5 treatments, the patient continued in the study until he/she achieved resolution or received 10 treatments. At the final visit, another 40 mL of venous blood was drawn.

The sequences of antigens to be used in the ELISPOT assay were chosen from the most common HPV types detected in the warts of patients previously recruited in our clinics (HPV 2a, 27, and 57) (13-15). They were derived using the predictive engines of MULTIPRED (22), hidden Markov models, and artificial neural network methods capable of predicting peptides that promiscuously bind multiple HLA alleles within one HLA supertype (A2, A3, or DR). The peptide sequences that contained HLA class I A2 hotspots, HLA class II DR hotspots, and were similar among HPV 2a, 27, and 57 were chosen: HPV 57-E1 (231-260 and 251-286), E2 (188-208), E4 (10-30), E6 (17-55), and L1 (380-412).

Peripheral blood mononuclear cells (PBMC) were isolated from heparinized whole blood and were cryopreserved. The PBMC samples collected pre-vaccination and post-vaccination were tested simultaneously to eliminate inter-assay variability. The ELISPOT assay protocol was modified from one previously described (23,24). Briefly, 96-well plates (MultiScreen-HA; Millipore, Bedford, Mass.) were coated with 5 μg/ml of primary anti-IFN-γ monoclonal antibody (Mabtech, Stockholm, Sweden) for capture in 50 μl/well of phosphate-buffered saline (PBS) and stored at 4° C. overnight. The plates were then washed four times with PBS and blocked using RPMI 1640 with 5% pooled human serum for 1 h at 37° C. Three hundred thousand PBMC were presented with 10 μM each of the HPV 57 peptides described above. Candin® skin test antigen (1:50 dilution) was also tested in addition to phytohemagglutinin (PHA) which served as the positive control. For the negative control, media with no-peptide was used. Human recombinant interleukin-2 (R&D Systems, Inc., Minneapolis, Minn.) (20 U/ml) was also added to all wells. After 40±2 h incubation at 37° C., the plate was washed four times with PBS containing 0.05% Tween-20. A total of 50 μl of secondary antibody (biotin-conjugated anti-IFN-γ monoclonal antibody) (Mabtech) in PBS at a final concentration of 1 μg/ml was added, and the plate was incubated for 2 h at 37° C. The plate was then washed four times with PBS containing 0.1% Tween-20. Avidin-bound biotinylated horseradish peroxidase H (Vectastain Elite ABC kit; Vector Laboratories, Inc., Burlingame, Calif.) was added for 1 h at 37° C. After four washings with PBS containing 0.1% Tween-20, stable diaminobenzene (Open Biosystems, Huntsville, Ala.) was added to develop the reaction. The plates were washed with distilled water three times and air-dried overnight. The spots formed by IFN-γ-secreting T-cells were counted with an automated ELISPOT analyzer (Cell Technology Inc., Jessup, Md.). The average spot-forming units (SFU) per antigen were calculated. A response was considered positive when the average SFU in wells with a given peptide was at least twice that of the average SFU in the no-peptide control wells (25).

### Results

### Safety of Investigational New Drug Candin®

The adverse events that occurred from treatments with Candin® were assessed, which included local and systemic reactions (Table 2). None of the patients experienced vaccine-related adverse events higher than Grade 2 (moderate). Typically, patients encountered injection site pain and mild erythema. There were 5 vaccine-unrelated adverse events, which included Grade 3 pain in the throat, pharynx, and larynx (Patient #2). This patient underwent tonsillectomy after his early termination from this study.

TABLE 2
Summary of Adverse Events

| | CTCAE Grade, Number of Events | | | |
|---|---|---|---|---|
| | (Number of Patients) | | | |
| Adverse Event | 1 | 2 | 3 | 4 |
| Hypertension | 1 (1)^{a} | — | — | — |
| Constitutional symptoms | 1 (1) | — | — | — |
| Constitutional symptoms - | 1 (1)^{a} | — | — | — |
| Other (Common cold) | | | | |
| Injection site reaction | 66 (14) | 8 (5) | — | — |
| Rash | 1 (1) | — | — | — |
| Dermatology - Other (Peeling) | 1 (1) | — | — | — |
| Hematoma | 1 (1) | — | — | — |
| Muscle weakness | 2 (2) | — | — | — |
| Pain - Extremity-limb | 5 (3) | — | — | — |
| Pain - Head/headache | 2 (1) | 1 (1) | — | — |
| Pain - Lymph node | 2 (1) | — | — | — |
| Pain - Throat/pharynx/larynx | — | 2 (1)^{a} | 1 (1)^{a} | — |
| Flu-like syndrome | 2 (2) | 1 (1) | — | — |

^{a}Vaccine-unrelated adverse events.

### Clinical Response

Eleven of 18 enrolled patients completed the study, of whom 9 (82%) had complete resolution of their treated warts, 1 (9%) had partial resolution, and 1 (9%) had no response (Table 3). Complete resolutions of the 1^{st} distant untreated warts were observed in 6 (75%) of 8 patients while those of the 2^{nd} distant warts were observed in 6 (100%) of 6 patients. None had 3^{rd} distant warts. The median number of injections required for complete resolution was 4 (range: 2-10).

Eight of the 11 patients who completed the study were available for the 6-month follow-up interview (Table 3). Six (86%) of the 7 complete responders who could be contacted did not experience recurrence of resolved warts. Interestingly, the partial responder (Patient #9) experienced complete resolution of treated and untreated distant warts 3 months after the final visit.

The remaining 7 of 18 enrolled patients dropped out of the study for the following reasons: study unrelated surgery (n=1), airfare expense (n=2), tired of study (n=1), car accident injury (n=1), and non-compliance (n=2). At the time of last injection, 5 (71%) had partial resolution after 2, 4, or 6 injections and 2 (29%) had no response after 1 or 2 injections. One of the partial responders reported to have achieved complete resolution after the last injection.

TABLE 3
Summary of Clinical Response

| | | | | Re- | |
|---|---|---|---|---|---|
| | | | Regression | gression | |
| | | Regression | of 1^{st} | of 2^{nd} | |
| Patient | Injections, | of Treated | Distant | Distant | 6-Month |
| No. | No. | Wart, % | Wart, % | Wart, % | Follow-Up |
| 3 | 8 | 100 | 100 | 100 | No |
| | | | | | recurrence |
| 5 | 4 | 100 | 100 | 100 | Could not |
| | | | | | be reached |
| 6 | 4 | 100 | 100 | 100 | No |
| | | | | | recurrence |
| 8 | 3 | 100 | 100 | 100 | Could not |
| | | | | | be reached |
| 9 | 10 | 36 | 59 | NA | Both warts |
| | | | | | resolved |
| 10 | 10 | 100 | 83^{a} | 100 | No |
| | | | | | recurrence |
| 11 | 4 | 100 | 100 | 100 | Recurrence |
| 12 | 9 | 100 | 100 | NA | No |
| | | | | | recurrence |
| 13 | 7 | 100 | NA | NA | No |
| | | | | | recurrence |
| 14 | 5 | 5 | NA | NA | Wart did |
| | | | | | not resolve |
| 18 | 2 | 100 | NA | NA | No |
| | | | | | recurrence |

Abbreviations:
NA, not applicable.
^{a}1^{st} distant wart completely resolved per 6-month follow-up interview.

### HPV DNA Typing

A total of 18 wart biopsies were obtained for PCR analysis and HPV-DNA was detected in 11 (61%) of 18 warts (Table 4). HPV 2 (44%) and HPV 57 (44%) were the most common types identified followed by HPV 27 (17%). Five (28%) warts were infected with HPV 2 and 27, 2 (11%) with HPV 2 and 57, and 1 (6%) with HPV 2, 27, and 57. All samples with detectable HPV-DNA were positive with the set of primers common for HPV types 2, 27, and 57 and with at least one of the multiplex primers with the exception of one sample (see Patient #3 in Table 4).

TABLE 4
Summary of HPV-DNA Typing Results

| Patient | Common | Multiplex Primers for | Beta- |
|---|---|---|---|
| No. | Primers | HPV 2, 27, or 57 | Globin |
| 1 | + | 2, 57 | + |
| 2 | − | — | + |
| 3 | + | — | + |
| 4 | − | — | + |
| 5 | + | 2, 27 | + |
| 6 | − | — | + |
| 7 | + | 2, 57 | + |
| 8 | − | — | + |
| 9 | + | 2, 57 | + |
| 10 | − | — | + |
| 11 | + | 2, 57 | + |
| 12 | + | 2, 27, 57 | + |
| 13 | + | 2, 27 | + |
| 14 | − | — | + |
| 15 | + | 2, 57 | + |
| 16 | + | 57 | + |
| 17 | + | 57 | + |
| 18 | − | — | + |

### Immune Response

The IFN-γ ELISPOT assay was performed on 10 of 11 patients who completed the study (Table 5). A sample from one of the patients was lost due to improper thawing. A positive response to HPV 57 L1-peptide (380-412) was most commonly detected (6 of 10 or 60%). One patient demonstrated a response to HPV 57 E4-peptide (10-30) (10%). No responses were detected to the other peptides and to the Candida antigen. All samples were positive against PHA positive control. In relation to the clinical response, 6 (67%) of 9 responders had a positive response to the L1 peptide suggesting L1-specific T-cells may play a role in wart regression. The one non-responder had no positive results. The immune responses against the HPV type 57 antigens were detectable in patients with positive and negative HPV-DNA results (Table 5).

TABLE 5
Summary of ELISPOT Assay Results

| | | | | | | | Antigen^{a} | | | |
|---|---|---|---|---|---|---|---|---|---|---|
| | Clinical | | | L1 | E1#1 | E1#2 | E2 | E4 | E6 | |
| Patient No. | Response | HPV Typing | Visit | (380-412) | (231-260) | (251-286) | (188-208) | (10-30) | (17-55) | Candida |
| 3 | Complete | 2, 27, or 57 | Pre- | — | — | — | — | — | — | — |
| | | | Post-#1 | — | — | — | — | ND | ND | ND |
| | | | Post-#2 | 4.1 | — | — | — | — | — | — |
| 5 | Complete | 2 and 27 | Pre- | — | — | — | — | — | — | — |
| | | | Post- | 2.0 | — | — | — | 2.3 | — | — |
| 8 | Complete | — | Pre- | — | — | ND | ND | ND | ND | ND |
| | | | Post- | — | — | — | — | — | — | — |
| 9 | Partial | 2 and 57 | Pre- | — | — | — | — | — | — | — |
| | | | Post-#1 | 2.1 | — | — | — | — | — | — |
| | | | Post-#2 | 2.1 | — | — | — | — | — | — |
| 10 | Complete | — | Pre- | — | — | — | — | — | — | — |
| | | | Post-#1 | 2.0 | — | — | — | — | — | — |
| | | | Post-#2 | — | — | — | — | — | — | — |
| 11 | Complete | 2 and 57 | Pre- | — | — | — | — | — | — | — |
| | | | Post- | — | ND | ND | ND | ND | ND | ND |
| 12 | Complete | 2, 27, and 57 | Pre- | — | — | — | — | — | — | — |
| | | | Post-#1 | 2.1 | — | — | — | — | — | — |
| | | | Post-#2 | — | — | — | — | — | — | — |
| 13 | Complete | 2 and 27 | Pre- | — | — | — | ND | ND | ND | ND |
| | | | Post-#1 | — | — | — | — | — | ND | ND |
| | | | Post-#2 | — | — | — | — | — | — | — |
| 14 | No response | — | Pre- | — | — | — | — | — | — | — |
| | | | Post- | — | — | — | — | — | — | — |
| 18 | Complete | — | Pre- | 2.5 | — | — | — | — | — | — |
| | | | Post- | — | — | — | — | — | — | — |

Abbreviations: Pre-, pre-vaccination; Post-, post-vaccination; ND, not done due to inadequate number of cells.
^{a}The numerical values represent the positivity index, i.e., the ratio between the number of sfu in wells with peptides over number of sfu in media control wells.

### Discussion

There are many different options available for the treatment of common warts. Nevertheless, due to the recalcitrant nature of warts, they present as a challenge to physicians and oftentimes cause frustration and significant discomfort in the patients. Immunotherapy using intralesional injection of Candida antigen is an attractive alternative that may induce long lasting immunity.

In this study, we evaluated the safety of Candin® in a phase I clinical trial, and examined immune responses to HPV 57 peptides. No vaccine-related adverse events greater than Grade 2 were observed. Likewise, none of the other studies that examined the use of Candida antigen for the treatment of warts have reported serious adverse events.¹³⁻¹⁸ As for the clinical response, 9 (82%) of 11 patients who completed the protocol achieved complete resolution of the treated wart in this study. This response rate is similar to that of previously published studies (13, 15, 16, 18). Furthermore, 75% of 1^{st} distant warts and 100% of 2nd distant warts completely resolved suggesting the induction of HPV-specific immunity. Similar frequencies of distant untreated wart resolution have also been reported in previous studies (13,16). When we evaluated the viral types, HPV 2, 27, or 57 DNA was detected in 11 (61%) of 18 warts biopsied, which is somewhat lower than 82% (120 of 146) (15), which we previously reported in the same patient population. This may be attributable to the small number of patients in the current study.

We examined the T-cell immune responses to six HPV 57 peptide antigens that were predicted to contain HLA-class I and class II epitopes using MULTIPRED, a computational system (22). The immune responses to HPV 57 L1-peptide (380-412) were the most common, and were detected in 6 (60%) of 10 patients examined (Table 5). All 6 patients demonstrated at least partial resolution of their wart(s) suggesting that L1-specific T-cells may be involved in wart regression. On the other hand, 1 patient (Patient #18) had a detectable response to the L1-peptide (380-412) prior to injections, which was not enhanced after vaccinations suggesting that there may be other key T-cell epitopes that were not examined in this study. To our knowledge this is the first demonstration of HPV 57 L1-specific immune response. One implication of this finding may be that incorporating the HPV 57 L1-peptide (380-412) with Candida antigens may represent a new treatment option for common warts that requires less than the median of 4 injections for complete resolution as was observed in this study. Nevertheless, a more definitive evidence that HPV 57 L1-specific T-cells play a role in wart regression needs to be obtained by comparing the T-cell responses between the Candin® treated group and the placebo group. Previously, we studied the role of T-cells in the same patient population, who were treated for common warts with Candida, mumps, or Trichophyton skin test antigens, using a proliferation assay.¹⁵ Extracts from wart tissues were used as antigens, and positive proliferation results were significantly associated with clinical response (p=0.002). Taken together these results support the role of T-cells in wart regression.

Traditionally, Candida, mumps, and Trichophyton were used as recall antigens to assess the adequacy of cell-mediated immunity in patients skin-tested for tuberculosis. No differences in responses were observed among these antigens when they were used for treating common warts (Candida, 59%; mumps, 51%; Trichophyton, 62%; p=0.48) (15). Because these antigens are derived from very different organisms, their immunologic determinants are unlikely to cross-react with HPV antigenic determinants. Since all of these recall antigens are derived from microorganisms, it may be possible that they work through pattern recognition receptors (PRRs).

In recent years, numerous PRRs, including Toll-like receptors (TLRs) (26,27), retinoic acid inducible gene-I like receptors (28), C-type lectin receptors (29,30), nucleotide-binding and oligomerization-like receptors (28), and scavenger receptors (31), have been described, and their role in inducing innate and potentially adaptive immunity has been studied. A number of unrelated findings support the notion that recall antigens may work through PRRs to impart anti-HPV effects. For example, imiquimod, FDA-approved topical treatment of genital warts, binds to TLR7 (32). In common warts, the levels of TLR3 and TLR9 are higher than in unaffected skin (33). When challenged with Candida albicans, mice deficient in myeloid differentiation factor 88 (MyD88), a universal TLR adaptor protein, demonstrated impaired survival compared to wild-type mice (34). Such impairment corresponded not only with deficient production of T helper type 1 (Th1) cytokines, but also with greatly diminished frequencies of IFN-γ producing CD4 and CD8 T-cells. Also, some evidence suggests that Candida albicans may work through TLR2 (34), TLR4 (35), and TLR6 (36). Mice deficient in TLR2 or TLR4 had impaired defense against Candida albicans infection. In TLR2-deficient mice (TLR2^{-/-}), in vitro production of tumor necrosis factor (TNF)-α and macrophage inhibitory protein (MIP)-2 by macrophages was reduced. In TLR4-defective C3H/HeJ mice, production of CXC chemokines KC and MIP-2 were decreased. The defects in both mouse strains resulted in decreased neutrophil recruitment. On the other hand, TLR6-deficient mice (TLR6^{-/-}) did not have increased susceptibility to Candida infections; rather, these mice demonstrated defective production of interleukin (IL)-10 and increased production of IFN-γ resulting in imbalanced Th1 and T helper type 2 (Th2) responses.

C-type lectin receptors include mannose receptors (37), mannose-binding lectins (38), DC-SIGN and related receptors (38), and β-glucan receptors (such as dectin-1) (39,40). Dectin-1 has an important role in the recognition of pathogenic fungi (Candida albicans, Aspergillus fumigatus, Pneumocystis carinii) and also has been shown to collaborate with TLR2 in the activation of NF-κB.⁴¹ The extracellular domain of dectin-2 has been shown to bind to the hyphal components of Candida albicans, Microsporum audouinii, and Trichophyton rubrum.⁴² Galectin-3, a lectin, interacts with β-1,2 mannosides on Candida albicans (43). Cambi et al. have demonstrated that DC-SIGN and mannose receptors specifically mediate the binding and internalization of Candida albicans by human dendritic cells (44). Another C-type lectin, mincle, plays an essential and non-redundant role in Candida-induced TNF-α induction in mouse macrophages (45). In summary, mannose receptor, dectin-1, dectin-2, galectin-3, DC-SIGN, mincle, and some TLRs have been shown to interact with Candida albicans. Dectin-1 is a particularly important candidate receptor which may interact with Candin® since its role in mediating the differentiation of human monocytes into dendritic cells has been demonstrated (17).

In short, Candin® is safe when injected into lesions as a treatment for warts, and the T-cells specific for HPV 57 L1 may play a role in wart regression. A larger phase II, placebo-controlled clinical trial is warranted, not only to confirm the clinical efficacy, but also to clarify the role of HPV-specific T-cells in clinical response.

SEQ ID NO: 1, Major capsid protein L1 of HPV
type 57:
MFCGLNDVNVCTISLQMAMWRPNESKVYLPPTPVSKVLSTDVYVTRTNVY
YHGGSSRLLTVGHPYYSIKKSGNNKVSVPKVSGYQYRVFHVKLPDPNKFG
LPDANLYDPDTQRLLWACVGVEVGRGQPLGVGISGHPYYNKQDDTENSHN
PDAADDGREYISMDYKQTQLFILGCKPPIGEHWSKGTTCSGSSAVGDCPP
LQFTNTTIEDGDMVETGFGALDFAALQSNKSDVPLDICTNICKYPDYLKM
AADPYGDSMFFSLRREQMFTRHFFNRGGSMGDALPDELYVKSSTVQTPGS
YVYTSTPSGSMVSSEQQLFNKPYWLRRAQGHNNGMCWGNRIFLTVVDTTR
STNVSLCATVTTETNYKASNYKEYLRHMEEYDLQFIFQLCKITLTPEIMA
YIHNMDARLLEDWNFGVPPPPSASLQDTYRYLQSQAITCQKPTPPKTPTD
PYATMTFWDVDLSESFSMDLDQFPLGRKFLLQRGATPTVSRKRAAATAAA
PTAKRKKVRR
SEQ ID NO: 2, Protein E4 of HPV type 57:
MGLLGRGRCRSGGVLFIIHPHLCLVPRPPPRTTTHYPLLDLLRPQSQPQP
QPQQQSRPHSRTPPRRHRVRHPSASGSSSDSSGNSPTLRGRSEKGRWSVK
TTGASVTLTAQTPGGATVTLTLCL
SEQ ID N0: 3, L1 380-412:
EYDLQFIFQLCKITLTPEIMAYIHNMDARLLED
SEQ ID NO: 4, E4 10-30:
RSGGVLFIIHPHLCLVPRPPP

### REFERENCES

1. Plasencia J M. Cutaneous warts: diagnosis and treatment. Prim Care. 2000; 27(2):423-434.
2. Rubben A, Kalka K, Spelten B, Grussendorf-Conen E I. Clinical features and age distribution of patients with HPV Feb. 27, 1957-induced common warts. Arch Dermatol Res. 1997; 289(6):337-340.
3. Kirnbauer R, Lenz P, Okun M M. Human Papillomavirus. In: Bolognia J, Jorizzo J, Rapini R, eds. Dermatology. 1st ed. London: Mosby; 2003:1217-1233.
4. Bacelieri R, Johnson S M. Cutaneous warts: an evidence-based approach to therapy. Am Fam Physician. 2005; 72(4):647-652.
5. Cobb M W. Human papillomavirus infection. J Am Acad Dermatol. 1990; 22(4):547-566.
6. Stern R S, Johnson M L, DeLozier J. Utilization of physician services for dermatologic complaints. The United States, 1974. Arch Dermatol. 1977; 113(8):1062-1066.
7. Baker G E, Tyring S K. Therapeutic approaches to papillomavirus infections. Dermatol Clin. 1997; 15(2):331-340.
8. Scott M, Nakagawa M, Moscicki A B. Cell-mediated immune response to human papillomavirus infection. Clin Diagn Lab Immunol. 2001; 8(2):209-220.
9. Harwood C A, Surentheran T, McGregor J M, et al. Human papillomavirus infection and non-melanoma skin cancer in immunosuppressed and immunocompetent individuals. J Med Virol. 2000; 61(3):289-297.
10. Bouwes Bavinck J N, Berkhout R J. HPV infections and immunosuppression. Clin Dermatol. 1997; 15(3):427-437.
11. Walder B K, Robertson M R, Jeremy D. Skin cancer and immunosuppression. Lancet. 1971; 2(7737):1282-1283.
12. Leigh I M, Glover M T. Skin cancer and warts in immunosuppressed renal transplant recipients. Recent Results Cancer Res. 1995; 139:69-86.
13. Johnson S M, Roberson P K, Horn T D. Intralesional injection of mumps or Candida skin test antigens: a novel immunotherapy for warts. Arch Dermatol. 2001; 137(4):451-455.
14. Clifton M M, Johnson S M, Roberson P K, Kincannon J, Horn T D. Immunotherapy for recalcitrant warts in children using intralesional mumps or Candida antigens. Pediatr Dermatol. 2003; 20(3):268-271.
15. Horn T D, Johnson S M, Helm R M, Roberson P K. Intralesional immunotherapy of warts with mumps, Candida, and Trichophyton skin test antigens: a single-blinded, randomized, and controlled trial. Arch Dermatol. 2005; 141(5):589-594.
16. Johnson S M, Horn T D. Intralesional immunotherapy for warts using a combination of skin test antigens: a safe and effective therapy. J Drugs Dermatol. 2004; 3(3):263-265.
17. Nisini R, Torosantucci A, Romagnoli G, et al. beta-Glucan of Candida albicans cell wall causes the subversion of human monocyte differentiation into dendritic cells. J Leukoc Biol. 2007; 82(5):1136-1142.
18. Maronn M, Salm C, Lyon V, Galbraith S. One-year experience with candida antigen immunotherapy for warts and molluscum. Pediatr Dermatol. 2008; 25(2):189-192.
19. Lei Y J, Gao C, An R, et al. Development of a multiplex PCR method for detecting and typing human papillomaviruses in verrucae vulgaris. J Virol Methods. 2008; 147(1):72-77.
20. Harwood C A, Spink P J, Surentheran T, et al. Degenerate and nested PCR: a highly sensitive and specific method for detection of human papillomavirus infection in cutaneous warts. J Clin Microbiol. 1999; 37(11):3545-3555.
21. Resnick R M, Cornelissen M T, Wright D K, et al. Detection and typing of human papillomavirus in archival cervical cancer specimens by DNA amplification with consensus primers. J Natl Cancer Inst. 1990; 82(18):1477-1484.
22. Zhang G L, Khan A M, Srinivasan K N, August J T, Brusic V. MULTIPRED: a computational system for prediction of promiscuous HLA binding peptides. Nucleic Acids Res. 2005; 33 (Web Server issue):W172-9.
23. Farhat S, Nakagawa M, Moscicki A B. Cell-mediated immune responses to human papillomavirus 16 E6 and E7 antigens as measured by interferon gamma enzyme-linked immunospot in women with cleared or persistent human papillomavirus infection. Int J Gynecol Cancer. 2009; 19(4):508-512.
24. Kim K H, Greenfield W, Shotts E, Nakagawa M. Detection of human papillomavirus type 16-specific T lymphocytes by a recombinant vaccinia virus-based enzyme-linked immunospot assay. Clin Vaccine Immunol. 2007; 14(4):362-368.
25. Kaul R, Dong T, Plummer F A, et al. CD8(+) lymphocytes respond to different HIV epitopes in seronegative and infected subjects. J Clin Invest. 2001; 107(10):1303-1310.
26. Takeda K, Kaisho T, Akira S. Toll-like receptors. Annu Rev Immunol. 2003; 21:335-376.
27. Smits E L, Ponsaerts P, Berneman Z N, Van Tendeloo V F. The use of TLR7 and TLR8 ligands for the enhancement of cancer immunotherapy. Oncologist. 2008; 13(8):859-875.
28. Creagh E M, O'Neill L A. TLRs, NLRs and RLRs: a trinity of pathogen sensors that co-operate in innate immunity. Trends Immunol. 2006; 27(8):352-357.
29. Weis W I, Taylor M E, Drickamer K. The C-type lectin superfamily in the immune system. Immunol Rev. 1998; 163:19-34.
30. Gordon S. Pattern recognition receptors: doubling up for the innate immune response. Cell. 2002; 111(7):927-930.
31. Areschoug T, Gordon S. Scavenger receptors: role in innate immunity and microbial pathogenesis. Cell Microbiol. 2009; 11(8):1160-1169.
32. Hemmi H, Kaisho T, Takeuchi O, et al. Small anti-viral compounds activate immune cells via the TLR7 MyD88-dependent signaling pathway. Nat. Immunol. 2002; 3(2):196-200.
33. Ku J K, Kwon H J, Kim M Y, et al. Expression of Toll-like receptors in verruca and molluscum contagiosum. J Korean Med Sci. 2008; 23(2):307-314.
34. Villamon E, Gozalbo D, Roig P, O'Connor J E, Fradelizi D, Gil M L. Toll-like receptor-2 is essential in murine defenses against Candida albicans infections. Microbes Infect. 2004; 6(1):1-7.
35. Netea M G, Van Der Graaf C A, Vonk A G, Verschueren I, Van Der Meer J W, Kullberg B J. The role of toll-like receptor (TLR) 2 and TLR4 in the host defense against disseminated candidiasis. J Infect Dis. 2002; 185(10):1483-1489.
36. Netea M G, van de Veerdonk F, Verschueren I, van der Meer J W, Kullberg B J. Role of TLR1 and TLR6 in the host defense against disseminated candidiasis. FEMS Immunol Med Microbiol. 2008; 52(1):118-123.
37. Stahl P D, Ezekowitz R A. The mannose receptor is a pattern recognition receptor involved in host defense. Curr Opin Immunol. 1998; 10(1):50-55.
38. Jack D L, Klein N J, Turner M W. Mannose-binding lectin: targeting the microbial world for complement attack and opsonophagocytosis. Immunol Rev. 2001; 180:86-99.
39. Brown G D, Gordon S. Immune recognition. A new receptor for beta-glucans. Nature. 2001; 413(6851):36-37.
40. Brown G D, Herre J, Williams D L, Willment J A, Marshall A S, Gordon S. Dectin-1 mediates the biological effects of beta-glucans. J Exp Med. 2003; 197(9):1119-1124.
41. Gantner B N, Simmons R M, Canavera S J, Akira S, Underhill D M. Collaborative induction of inflammatory responses by dectin-1 and Toll-like receptor 2. J Exp Med. 2003; 197(9):1107-1117.
42. Sato K, Yang X L, Yudate T, et al. Dectin-2 is a pattern recognition receptor for fungi that couples with the Fc receptor gamma chain to induce innate immune responses. J Biol Chem. 2006; 281(50):38854-38866.
43. Jouault T, El Abed-El Behi M, Martinez-Esparza M, et al. Specific recognition of Candida albicans by macrophages requires galectin-3 to discriminate Saccharomyces cerevisiae and needs association with TLR2 for signaling. J Immunol. 2006; 177(7):4679-4687.
44. Cambi A, Netea M G, Mora-Montes H M, et al. Dendritic cell interaction with Candida albicans critically depends on N-linked mannan. J Biol Chem. 2008; 283(29):20590-20599.
45. Wells C A, Salvage-Jones J A, Li X, et al. The macrophage-inducible C-type lectin, mincle, is an essential component of the innate immune response to Candida albicans. J Immunol. 2008; 180(11):7404-7413.

All patents, patent documents, and non-patent references cited herein are incorporated by reference.

What is claimed:

1. A pharmaceutical composition comprising a polypeptide comprising (a) L1 380-412 (SEQ ID NO:3) or a fragment of at least 8 residues of SEQ ID NO:3 or (b) E4 10-30 (SEQ ID NO:4) or a fragment of at least 8 residues of E4 10-30 (SEQ ID NO:4), wherein the polypeptide does not comprise L1 (SEQ ID NO:1) or E4 (SEQ ID NO:2), wherein the composition is immunogenic in humans.

2. The pharmaceutical composition of claim 1 wherein the polypeptide is a polypeptide of 8-100 amino acid residues.

3. The pharmaceutical composition of claim 1 wherein the polypeptide is a polypeptide of 8-50 amino acid residues.

4. The pharmaceutical composition of claim 1 wherein the composition is capable of inducing a delayed-type hypersensitivity response.

5. The pharmaceutical composition of claim 1 wherein the polypeptide comprises SEQ ID NO:3 or a fragment of at least 8 residues of SEQ ID NO:3.

6. The pharmaceutical composition of claim 1 wherein the polypeptide comprises SEQ ID NO:4 or a fragment of at least 8 residues of SEQ ID NO:4.

7. The composition of claim 1 wherein the composition is capable of treating an epithelial tumor caused by a human papilloma virus.

8. The composition of claim 7 wherein the composition is capable of treating a benign epithelial tumor caused by a human papilloma virus.

9. The composition of claim 1 wherein the composition comprises an antigen not found in a human papilloma virus.

10. The composition of claim 9 wherein the composition comprises candida antigen, trichophyton antigen, or mumps antigen.

11. The pharmaceutical composition of claim 1 wherein the composition is sterile and suitable for injection in a human intradermally, intramuscularly, or subcutaneously, or suitable for vaginal or nasal inoculation.

12. The pharmaceutical composition of claim 11 wherein the composition is sterile and suitable for injection in a human intradermally, intramuscularly, or subcutaneously.

13. The pharmaceutical composition of claim 1 further comprising at least one cytokine or colony stimulating factor.

14. The pharmaceutical composition of claim 13 wherein the composition comprises granulocyte colony stimulating factor, granulocyte macrophage colony stimulating colony stimulating factor, interferon alpha, interferon beta, interferon gamma, interleukin-2, or interleukin-12.

15. The pharmaceutical composition of claim 5 wherein the polypeptide is amidated at its C-terminus.

16. A pharmaceutical composition comprising:
(a) an antigen not found in human papilloma virus; and
(b) (i) a polypeptide comprising human papillomavirus (HPV) L1 (SEQ ID NO:1) or an antigenic fragment of SEQ ID NO:1 or (b) (ii) a polypeptide comprising HPV E4 (SEQ ID NO:2) or an antigenic fragment of SEQ ID NO:2; wherein the composition is capable of treating a benign epithelial tumor caused by a human papilloma virus;

17. The pharmaceutical composition of claim 16 wherein the polypeptide comprises. SEQ ID NO:3 or a fragment of at least 8 residues of SEQ ID NO:3.

18. A method of treating an epithelial tumor in a human comprising:
inoculating the human with a pharmaceutical composition comprising a polypeptide comprising (a) L1 380-412 (SEQ ID NO:3) or a fragment of at least 8 residues of SEQ ID NO:3 or (b) E4 10-30 (SEQ ID NO:4) or a fragment of at least 8 residues of SEQ ID NO:4, wherein the polypeptide does not comprise L1 (SEQ ID NO:1) or E4 (SEQ ID NO:2), wherein the composition is immunogenic in humans; wherein the epithelial tumor is caused by a human papilloma virus.

19. The method of claim 18 wherein the step of inoculating the pharmaceutical composition induces a delayed-type hypersensitivity response in the human.

20. The method of claim 18 further comprising inoculating the human with at least one antigen not found in a human papilloma virus; wherein the antigen induces a delayed-type hypersensitivity response in the human.

21. The method of claim 18 wherein the polypeptide is a polypeptide of 8-100 amino acid residues.

22. The method of claim 18 wherein the epithelial tumor is a benign epithelial tumor.

23. The method of claim 18 wherein the epithelial tumor is a cervical carcinoma tumor, an oral carcinoma tumor, or a vaginal carcinoma tumor.

24. The method of claim 18 wherein the method comprises inoculating the human with the composition at least twice, wherein the two inoculations are spaced at least one week apart.

25. The method of claim 18 wherein the polypeptide is amidated at its C-terminus.

26. A kit comprising (a) a pharmaceutical composition, suitable for inoculation of a human, comprising an antigen capable of inducing a delayed type hypersensitivity response in humans, wherein the composition is immunogenic, wherein the antigen is not found in a human papilloma virus; and (b) a pharmaceutical composition, suitable for inoculation of a human, comprising (i) a polypeptide comprising human papillomavirus (HPV) L1 (SEQ ID NO:1) or an antigenic fragment of SEQ ID NO:1 or (ii) a polypeptide comprising HPV E4 (SEQ ID NO:2) or an antigenic fragment of SEQ ID NO:2.


SEQUENCE LISTING (4 sequences)

SEQ ID NO: 1 - protein, 510 aa, Human papillomavirus type 57
MFCGLNDVNVCTISLQMAMWRPNESKVYLPPTPVSKVLSTDVYVTRTNVYYHGGSSRLLTVGHPYYSIKKSGNNKVSVPKVSGYQYRVFHVKLPDPNKFGLPDANLYDPDTQRLLWACVGVEVGRGQPLGVGISGHPYYNKQDDTENSHNPDAADDGREYISMDYKQTQLFILGCKPPIGEHWSKGTTCSGSSAVGDCPPLQFTNTTIEDGDMVETGFGALDFAALQSNKSDVPLDICTNICKYPDYLKMAADPYGDSMFFSLRREQMFTRHFFNRGGSMGDALPDELYVKSSTVQTPGSYVYTSTPSGSMVSSEQQLFNKPYWLRRAQGHNNGMCWGNRIFLTVVDTTRSTNVSLCATVTTETNYKASNYKEYLRHMEEYDLQFIFQLCKITLTPEIMAYIHNMDARLLEDWNFGVPPPPSASLQDTYRYLQSQAITCQKPTPPKTPTDPYATMTFWDVDLSESFSMDLDQFPLGRKFLLQRGATPTVSRKRAAATAAAPTAKRKKVRR

SEQ ID NO: 2 - protein, 124 aa, Human papillomavirus type 57
MGLLGRGRCRSGGVLFIIHPHLCLVPRPPPRTTTHYPLLDLLRPQSQPQPQPQQQSRPHSRTPPRRHRVRHPSASGSSSDSSGNSPTLRGRSEKGRWSVKTTGASVTLTAQTPGGATVTLTLCL

SEQ ID NO: 3 - protein, 33 aa, Human papillomavirus type 57
EYDLQFIFQLCKITLTPEIMAYIHNMDARLLED

SEQ ID NO: 4 - protein, 21 aa, Human papillomavirus type 57
RSGGVLFIIHPHLCLVPRPPP